(19) United States
(12) Patent Application Publication
(10) Pub. No.: US 2010/0098809 A1
(43) Pub. Date: Apr. 22, 2010

(54) GENETIC MARKER WEIGHT MANAGEMENT

(75) Inventors: Lewis H. Bender, Redding, CT (US); Colleen Draper, Stoneham, MA (US); Gary Breton, Marlborough, MA (US); Leon Wilkins, North Andover, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Interleukin Genetics, Inc., Waltham, MA (US)

(21) Appl. No.: 12/466,834

(22) Filed: May 15, 2009

Related U.S. Application Data
(60) Provisional application No. 61/053,888, filed on May 16, 2008.

(51) Int. Cl. A23L 1/307; G06F 19/00; C12Q 1/68

(52) U.S. Cl. 426/87; 702/19; 435/6

(57) ABSTRACT

A system and method for facilitating personal weight management based on genetic markers. Genetic samples can be analyzed and genotype patterns can be identified and provided to a user. Wellness information, including macronutrient requirements and exercise requirements, based on genotype patterns can be generated. Genotype patterns and wellness information can be stored, transmitted and displayed via computer networks and the internet. Existing third party diets, meal plans, and/or fitness programs can be recommended based on the genotype patterns. Individuals can use nutritional tracking software and/or exercise metric programs coupled with their genetic information to make adjustments to their diet and/or exercise to lose weight. Individuals can purchase nutrition products based on their genotype information.

### CROSS-REFERENCE TO RELATED ACTIONS

This application claims the benefit of U.S. Provisional Application No. 61/053,888, filed on May 16, 2008, the disclosure of which is hereby incorporated by reference in its entirety.

### BACKGROUND

According to a report published in 1998 by the World Health Organization (WHO), obesity has reached epidemic proportions worldwide: about 1.7 billion people worldwide are overweight and 300 million of them are obese. In the U.S. approximately 127 million adults are overweight and 69 million are obese. Obese subjects are at increased risk of developing one or more serious medical conditions including diabetes, heart disease, high blood pressure and high blood cholesterol. The prevalence of obesity has more than doubled in the past 25 years and now reaches 31% among U.S. adults aged 20 years and older. Higher rates of obesity are seen among African-Americans and Hispanic Americans, especially among women (30% to 50%).

The increase in the prevalence of obesity observed worldwide in the past decades has occurred in a changing environment characterized by a progressive reduction of physical activity level and the abundance of highly palatable foods. The WHO Report identified these changes as the two principal modifiable characteristics of modern lifestyle promoting the development of obesity. However, despite the fact that people are exposed to the same environment, not everyone is becoming obese, suggesting a role for an individual's genetic profile in the development of weight management issues. That is, genetics determines an individual's susceptibility to become obese when exposed to a unfavorable environment as well as the way he/she can respond to diet and exercise.

Accordingly, there is a need for a means for establishing and computing a personalized weight loss program that considers a person's genetic susceptibility to obesity in order to improve weight loss and weight maintenance outcomes relative to a similar program not taking into account genetic information. There is a need for a means for receiving a subject's metabolic genotype, calculating a response including diet and/or exercise, and providing the response to the subject.

### SUMMARY

In general, in an aspect, the invention provides a method for transforming a dietary program of a person as a function of their genotype pattern. In another aspect, the invention provides a computerized method for providing wellness information to a user, including receiving a genetic sample, determining a genotype pattern from the genetic sample, generating, on at least one computer, a wellness report based on the genotype pattern, and sending the wellness report to the user.

Implementations of the invention may include one or more of the following features. Receiving the genetic sample can include receiving at least one consent form and a packet containing at least one genetic sample. The packet and the consent form can include matching bar-code information. The genetic sample can be a brush containing a tissue sample extracted from inside the user's cheek. Determining the genotype pattern can include identifying the genotype with respect to at least one metabolic gene or gene variation. Determining the genotype pattern can include identifying the genotype with respect to at least one of FABP2 (rs1799883), PPARG (rs1801282), ADRB3 (rs4994), ADRB2 (rs1042713), or ADRB2 (rs1042714). The wellness report can include at least one logo representative of the genotype pattern. The wellness report can be provided to the user via a computer network.

In general, in another aspect, the invention provides an article of manufacture including a nutritional product suitable to be consumed in a diet such as a balanced diet, a low-fat diet, or a low-carb diet, and a genotype pattern logo can be disposed on the nutritional product, such that the genotype pattern logo represents a genotype that is used to predict a person's responsiveness to at least one of the diets.

Implementations of the invention may include one or more of the following features. The genotype pattern logo can include a background component and a tagline component. The genotype pattern logo can be associated with the person's genetic polymorphism pattern consisting of at least one metabolic gene or gene variant. The genotype pattern logo be associated with the person's genetic polymorphism pattern with respect to one or more of FABP2 (rs1799883), PPARG (rs1801282), ADRB3 (rs4994), ADRB2 (rs1042713), or ADRB2 (rs1042714).

In general, in another embodiment, the invention provides a method of providing genetic weight management information to a user, including obtaining a tissue sample from the user, transforming the tissue sample into metabolic genotype pattern information, and providing the genotype pattern information to the user.

Implementations of the invention may include one or more of the following features. Generating a wellness report, such that the wellness report can include diet and exercise recommendations. Providing the genotype pattern information can include providing a genotype pattern logo. Transforming the tissue sample can include identifying the user's genetic polymorphism pattern with respect to one or more of the following: FABP2 (rs1799883), PPARG (rs1801282), ADRB3 (rs4994), ADRB2 (rs1042713), or ADRB2 (rs1042714). Providing the user a kit including a consent form, a plurality of brushes, a drying stand, a packet, and a mailing envelope. The user can rub at least one brush against the inside of their cheek. The brush can be placed in the drying stand for approximately 15 minutes to dry, and the brush can be sealed in the packet and mailed to a test facility. The consent form and the packet can contain identical bar code information.

In general, in another aspect, the invention provides a computer network including a data storage device configured to store wellness information, at least one display device configured to receive and display information from and to a user, and a processor programmed to receive a genotype pattern information from the user, and provide wellness information as a function of the genotype pattern information to the user via the display.

Implementations of the invention may include one or more of the following features. The wellness information can include recipes, exercise recommendations, and/or recommendations for dietary supplements.

In accordance with implementations of the invention, one or more of the following capabilities may be provided. Genetic samples can be collected and analyzed. Genotype patterns based on the genetic samples can be identified and provided to a user. Macronutrient requirements based on genotype patterns can be established. Exercise requirements based on genotype patterns can be established. A personal diet and exercise plan can be provided to a user. Existing third party diets, meal plans, and/or fitness programs can be recommended based on the genotype patterns. Genotype patterns can be stored, transmitted and displayed via computer networks and the internet. The preexisting dietary and exercise programs of a person can be transformed based on their genotype pattern. Individuals can use nutritional tracking software and/or exercise metric programs coupled with their genetic information to make adjustments to their diet and/or exercise to lose weight. Individuals can purchase nutrition products based on their genotype information.

These and other capabilities of the invention, along with the invention itself, will be more fully understood after a review of the following figures, detailed description, and claims.

### BRIEF DESCRIPTION OF THE FIGURES

FIG. 1 is an exemplary block diagram for assigning a macronutrient category and/or an exercise category based on genotype patterns.

FIG. 2 is an exemplary network diagram for a genetic marker weight management network.

FIG. 2A is an exemplary flow diagram for obtaining a tissue sample.

FIG. 3 is a process flow diagram for aligning each of six genotype patterns to an appropriate diet and exercise program.

FIGS. 4A-C are exemplary logos which correspond to at least one genetic marker.

### DETAILED DESCRIPTION OF PREFERRED EMBODIMENTS

Embodiments of the invention provide techniques for weight management through the identification of genotype markers. The techniques can include providing wellness information (e.g., weight management information) such as dietary information (i.e. food and dietary supplements), exercise information, or both. These systems and methods are exemplary, however, and not limiting of the invention as other implementations in accordance with the disclosure are possible.

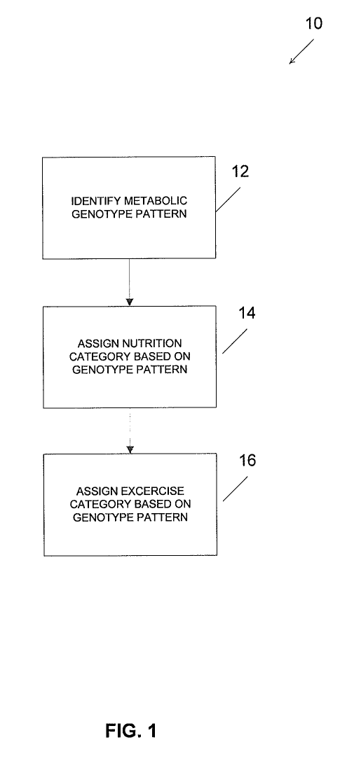

Referring to FIG. 1, a process 10 for genetic marker weight management is shown. The process 10, however, is exemplary only and not limiting. The process 10 may be altered, e.g., by having stages added, removed, or rearranged.

At stage 12, a metabolic genotype pattern can be identified. As disclosed in related applications U.S. Provisional Application No. 61/053,888, and co-pending U.S. application Ser. No. 12/466,614 filed on May 15, 2009 with Attorney Docket No. 24299-537001US, tests to determine an individual's “metabolic genotype,” can involve determining an individual's genotype for one or more (e.g., 2, 3, 4, etc) metabolic genes. The results of such metabolic genotyping can be used to predict a subject's responsiveness to relative amounts of macronutrients and calorie restriction in the diet, with or without exercise, for weight loss. These disclosures are incorporated by reference in their entirety.

At stages 14 and 16, an appropriate therapeutic/dietary regime or lifestyle recommendation for a subject can be assigned. For example, according to some embodiments, the subject with a combined genotype of FABP2 (rs1799883) 1.1, PPARG (rs1801282) 1.1, ADRB2 (rs1042714) 1.1, and ADRB2 (rs1042713) 2.2, and ADRB3 (rs4994) 1.1 is predicted to be responsive to: a low fat or low carbohydrate, calorie-restricted diet; regular exercise; or both.

According to some embodiments, a subject with a combined genotype of one of FABP2 (rs1799883) 1.1 or 1.2 and PPARG (rs1801282) 1.1, and additionally one of ADRB2 (rs1042714) 1.1, 1.2, or 2.2 in combination with ADRB2 (rs1042713) 2.2 and ADRB3 (rs4994) 1.1 is predicted to be responsive to: a low fat, calorie-restricted diet; regular exercise; or both.

According to some embodiments, a subject with a combined genotype of one of PPARG (rs1801282) 1.2 or 2.2 and/or one of ADRB2 (rs1042714) 1.2 or 2.2, in combination with ADRB2 (rs1042713) 2.2 and ADRB3 (rs4994) 1.1 is predicted to be responsive to: a low carbohydrate, calorie-restricted diet; regular exercise; or both.

According to some embodiments, a subject with a combined genotype of one of PPARG (rs1801282) 1.2 or 2.2 and one of FABP2 (rs1799883) 1.1 or 1.2, in combination with ADRB2 (rs1042713) 2.2 and ADRB3 (rs4994) 1.1 is predicted to be responsive to: a low carbohydrate, calorie-restricted diet; regular exercise; or both.

According to some embodiments, a subject with a combined genotype of FABP2 (rs1799883) 1.1 and PPARG (rs1801282) 1.1, in combination with one of ADRB2 (rs1042713) 1.2 or 1.1 or one of ADRB3 (rs4994) 1.2 or 2.2 is predicted to be responsive to a low fat or low carbohydrate, calorie-restricted diet. According to some embodiments, the subject is further predicted to be less responsive to regular exercise.

According to some embodiments, a subject with a combined genotype of one of FABP2 (rs1799883) 1.1 or 1.2 and PPARG (rs1801282) 1.1, in combination with one of ADRB2 (rs1042714) 1.1, 1.2, or 2.2 and either one of ADRB2 (rs1042713) 1.1 or 1.2 or one of ADRB3 (rs4994) 1.2 or 2.2 is predicted to be responsive to: a low fat, calorie-restricted diet. According to some embodiments, the subject is further predicted to be less responsive to regular exercise.

According to some embodiments, a subject with a combined genotype of one of PPARG (rs1801282) 1.2 or 2.2 and/or one of ADRB2 (rs1042714) 1.2 or 2.2, in combination with one of ADRB2 (rs1042713) 1.1 or 1.2 or one of ADRB3 (rs4994) 1.2 or 2.2 is predicted to be responsive to: a low carbohydrate, calorie-restricted diet. According to some embodiments, the subject is further predicted to be less responsive to regular exercise.

According to some embodiments, a subject with a combined genotype of one of PPARG (rs1801282) 1.2 or 2.2 and one of FABP2 (rs1799883) 1.1 or 1.2, in combination with one of ADRB2 (rs1042713) 1.1 or 1.2 or one of ADRB3 (rs4994) 1.2 or 2.2 is predicted to be responsive to: a low carbohydrate, calorie-restricted diet. According to some embodiments, the subject is further predicted to be less responsive to regular exercise.

According to some embodiments, a subject with a combined genotype of FABP2 (rs1799883) 1.1 (Ala/Ala or G/G), PPARG (rs1801282) 1.1 (Pro/Pro or C/C), ADRB2 (rs1042714) 1.1 (Gln/Gln or C/C), and ADRB2 (rs1042713) 2.2 (Arg/Arg or A/A), and ADRB3 (rs4994) 1.1 (Trp/Trp or T/T) is predicted to be responsive to: a low fat or low carbohydrate, calorie-restricted diet; regular exercise; or both.

According to some embodiments, a subject with a combined genotype of one of FABP2 (rs1799883) 1.1 (Ala/Ala or G/G) or 1.2 (Ala/Thr or G/A) and PPARG (rs1801282) 1.1 (Pro/Pro or C/C), and additionally one of ADRB2 (rs1042714) 1.1 (Gln/Gln or C/C), 1.2 (Gln/Glu or C/G), or 2.2 (Glu/Glu or G/G) in combination with ADRB2 (rs1042713) 2.2 (Arg/Arg or A/A) and ADRB3 (rs4994) 1.1 (Trp/Trp or T/T) is predicted to be responsive to: a low fat, calorie-restricted diet; regular exercise; or both.

According to some embodiments, a subject with a combined genotype of one of PPARG (rs1801282) 1.2 (Pro/Ala (C/G) or 2.2 (Ala/Ala or G/G) and/or one of ADRB2 (rs1042714) 1.2 (Gln/Glu or C/G) or 2.2 (Glu/Glu or G/G), in combination with ADRB2 (rs1042713) 2.2 (Arg/Arg or A/A) and ADRB3 (rs4994) 1.1 (Trp/Trp or T/T) is predicted to be responsive to: a low carbohydrate, calorie-restricted diet; regular exercise; or both.

According to some embodiments, a subject with a combined genotype of one of PPARG (rs1801282) 1.2 (Pro/Ala or C/G) or 2.2 (Ala/Ala or G/G) and one of FABP2 (rs1799883) 1.1 (Ala/Ala or G/G) or 1.2 (Ala/Thr or G/A), in combination with ADRB2 (rs1042713) 2.2 (Arg/Arg or A/A) and ADRB3 (rs4994) 1.1 (Trp/Trp or T/T) is predicted to be responsive to: a low carbohydrate, calorie-restricted diet; regular exercise; or both.

According to some embodiments, a subject with a combined genotype of FABP2 (rs1799883) 1.1 (Ala/Ala or G/G) and PPARG (rs1801282) 1.1 (Pro/Pro or C/C), in combination with one of ADRB2 (rs1042713) 1.2 (Gly/Arg or G/A) or 2.2 (Arg/Arg or A/A) or one of ADRB3 (rs4994) 1.2 (Arg/Trp or T/C) or 2.2 (Arg/Arg or C/C) is predicted to be responsive to a low fat or low carbohydrate, calorie-restricted diet. According to some embodiments, the subject is further predicted to be less responsive to regular exercise.

According to some embodiments, a subject with a combined genotype of one of FABP2 (rs1799883) 1.1 (Ala/Ala or G/G) or 1.2 (Ala/Thr or G/A) and PPARG (rs1801282) 1.1 (Pro/Pro or C/C), in combination with one of ADRB2 (rs1042714) 1.1 (Gln/Gln or C/C), 1.2 (Gln/Glu or C/G), or 2.2 (Glu/Glu or G/G) and either one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low fat, calorie-restricted diet. According to some embodiments, the subject is further predicted to be less responsive to regular exercise.

According to some embodiments, a subject with a combined genotype of one of PPARG (rs1801282) 1.2 (Pro/Ala or C/G) or 2.2 (Ala/Ala or G/G) and/or one of ADRB2 (rs1042714) 1.2 (Gln/Glu or C/G) or 2.2 (Glu/Glu or G/G), in combination with one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low carbohydrate, calorie-restricted diet. According to some embodiments, the subject is further predicted to be less responsive to regular exercise.

According to some embodiments, a subject with a combined genotype of one of PPARG (rs1801282) 1.2 (Pro/Ala or C/G) or 2.2 (Ala/Ala or G/G) and one of FABP2 (rs1799883) 1.1 (Ala/Ala or G/G) or 1.2 (Ala/Thr or G/A), in combination with one of ADRB2 (rs1042713) 1.1 (Gly/Gly or G/G) or 1.2 (Gly/Arg or G/A) or one of ADRB3 (rs4994) 1.2 (Trp/Arg or T/C) or 2.2 (Arg/Arg or C/C) is predicted to be responsive to: a low carbohydrate, calorie-restricted diet. According to some embodiments, the subject is further predicted to be less responsive to regular exercise.

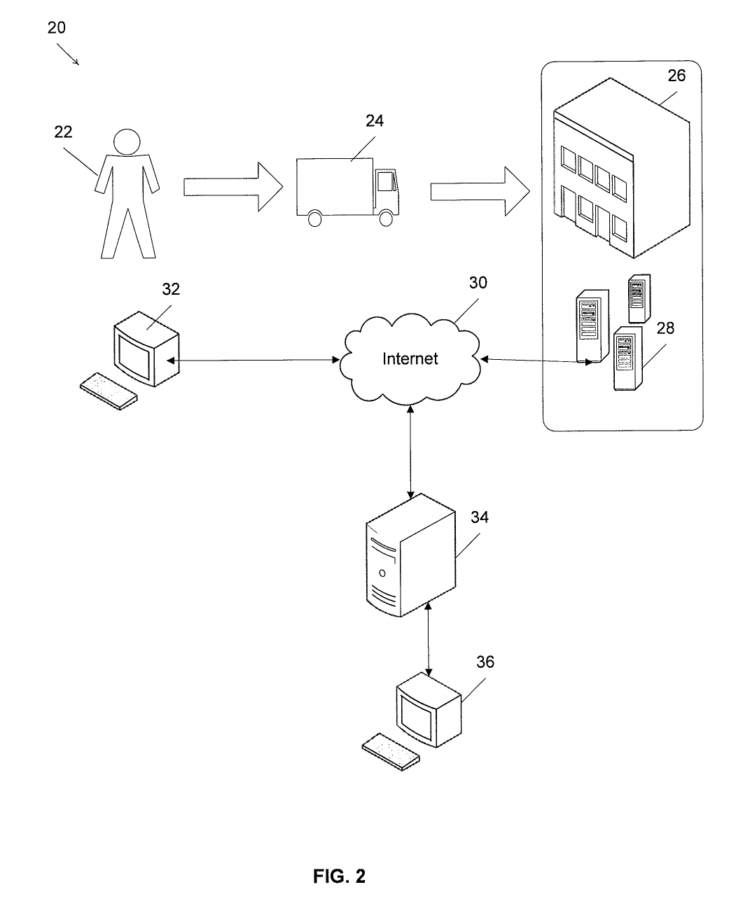

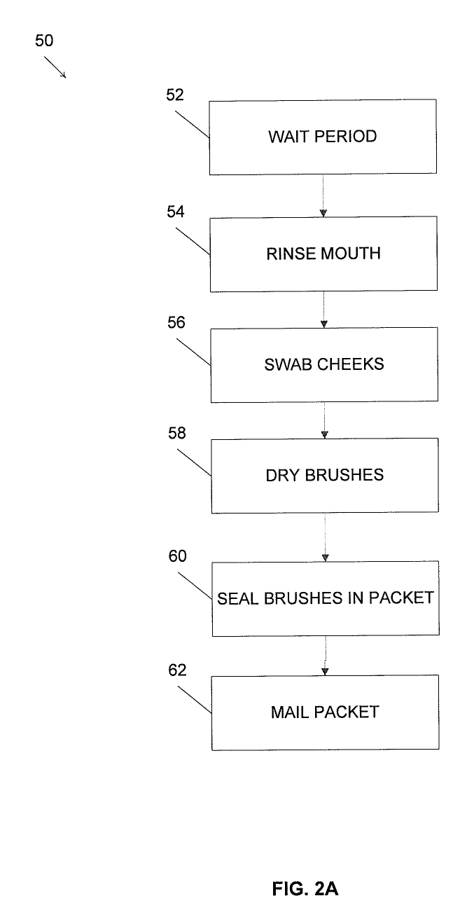

In operation, referring to FIG. 2, with further reference to FIG. 1, a genetic marker weight management network 20 is shown. The network 20, however, is exemplary only and not limiting. The network 20 may be altered, e.g., by having components added, removed, or rearranged.

The network 20 includes a user 22, a delivery component 24, a test facility 26 with at least one computer 28, connectivity to a network 30 (e.g., WAN, LAN, Internet), at least one user terminal 32 (e.g., personal computer, PDA, cell phone), a third party server 34 and third party administrator terminal 36.

In this example, the user 22 is seeking genetic marker weight management information. The user 22 provides a genetic sample. Any sample containing DNA is suitable (e.g., a buccal swab, blood sample, saliva sample). In an embodiment, the genetic sample collection is in the form of a kit which can be purchased off-the-shelf or provided via mail order. The kit can include information and bar-coded consent forms, at least two brushes, a drying stand, bar-coded packets, informational DVD, and return mailing envelopes. Referring to FIG. 2A, a process for obtaining a tissues sample 50 is shown. At stage 52, the user 22 is instructed to wait at least 2 hours after eating or brushing their teeth before continuing with the process 50. At stage 54, the user 22 rinses their mouth with water. At stage 56, the user 22 places a brush (i.e., swab) against the inside of their cheek and twists the brush while rubbing up and down firmly at least 20 times. At stage 58 the first brush can be placed in the drying stand while stage 56 is repeated with a second brush on the inside of the other cheek. Both brushes should be allowed to dry in the drying stand for at least 15 minutes. At stage 60, the brushes can be sealed in the bar-coded packet. The bar coding on the consent form and the packet helps to ensure the tissue sample is properly indentified during subsequent analysis. In an embodiment, the entire kit includes a bar-code such that the user's 22 information is collected and correlated at the time the kit is ordered. In an embodiment, the user 22 can access a website to enter their personal information, as well as an ID number associated with the bar-coded packet and consent forms. At stage 62, the consent forms and the used brushes (i.e., with the tissue sample) can be placed in the mail.

Referring back to FIG. 2, the user's 22 tissue (i.e. genetic) sample can be forwarded to the test facility 26 via the delivery component 24 (e.g., mail carriers, hand delivery). The sample collection may also be performed at a third party facility or at the test facility 26 directly. The test facility 26, and the computers 28, are configured to determine a genotype pattern for the user 22 based on the genetic sample. The user's 22 genotype pattern, as well as other personal information, can be stored on the computers 28. In general, the computers 28, 32, 34, 36 include processors, memory, operating systems, input and output devices as known in the art. For example, the computers 28, 32, 34, 36 can be personal computers and/or servers based on Intel® processing structures and running Microsoft Windows® operating systems. The computers 28, 32, 34, 36 can be configured interpret instructions via a computer-readable medium such as floppy disks, conventional hard disks, CD-ROMS, DVDs, Flash ROMS, nonvolatile ROM, and RAM. The computers 28, 32, 34, 36 can be configured to generate and store wellness information including nutritional guidelines and/or exercise suggestions based on genotype pattern information.

In operation, in an embodiment, the wellness information is stored on the computers 28 at the test facility 26. After the user's 22 genetic sample is received and processed, the genotype pattern information and corresponding wellness information can be produced in the form of a hardcopy report and mailed to the user 22. The wellness report can include information and recommendations regarding lifestyle choices such as nutritional guidelines (e.g., diet and supplements) and physical activity guidelines. The genotype pattern information and corresponding wellness information can also be electronically distributed via the network 30 to the user terminal 32 such as through an email message, a personal account in a website application, or delivered via third party networking applications (e.g., social networking sites).

In an embodiment, the computers 28 are configured to communicate with the third party servers 34. Portions of the user's personal information, weight management information and genotype pattern information can be formatted (e.g., binary, XML, text delimited) on the computers 28 such that they can be received by a third party server 34. Other communication protocols may also be used. For example, the third party server 34 can be an established weight loss website such as WeightWatchers.com®, or other similar applications. In general, these applications include datasets of wellness information such as food/recipes, fitness/health items, or both. When the application user logs on to their account, they can be prompted to enter their genotype pattern information. For example, the user 22 can identify that they are a “Pattern X” genotype (e.g., Carb-curber, Balanced, or other corresponding description) via a textbox, radio button, combo-box, or similar objects in a GUI. The third party server can be configured to present food/recipes, dietary supplements, and fitness/health items appropriate for the genotype. In an embodiment, the existing wellness information (e.g., the preexisting dietary and exercise programs available or suggested to the user) can be transformed as a function of the genotype pattern information. The wellness information can persist on the computers 28 and can be provided to the servers 34 as required. The genotype pattern wellness information can also be stored on the third party servers 34 and maintained by a third party administrator 36. That is, the administrator 36 can create datasets to correlate their existing wellness information to corresponding genotype pattern information. In an embodiment, the user's 22 genotype pattern information can be incorporated into the third party application, and the genotype pattern wellness recommendations can be based on corresponding subsets of the third party's food/recipes and fitness/health items.

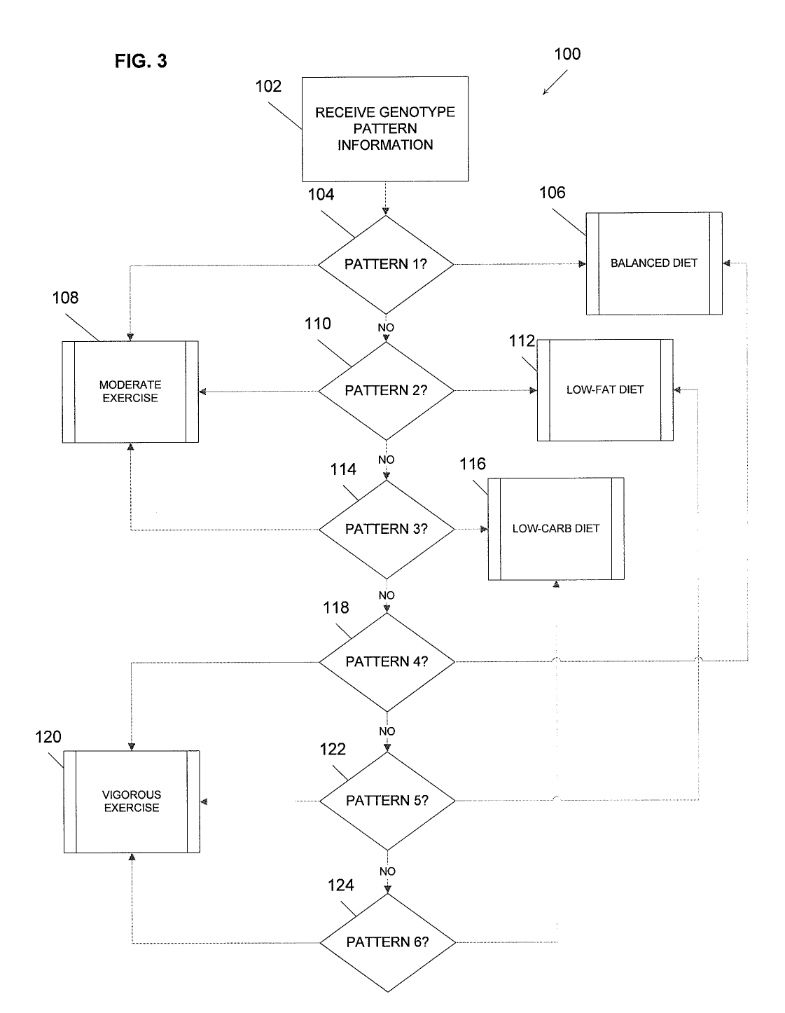

In operation, referring to FIG. 3, with further reference to FIGS. 1 and 2, a process flow diagram 100 for selecting a diet and exercise program using the genotype marker weight management network 20 includes the stages shown. The process 100, however, is exemplary only and not limiting. The process 100 may be altered, e.g., by having stages added, removed, or rearranged.

In an embodiment, the decisions and datasets in the process 100 can comprise computer-executable instructions stored on computer-readable medium and configured to be executed on computing device such as the computers 28, the user terminal 32, the third party server 34, and the administrator terminal 36. The process 100 can be combined and accessed via other applications (e.g., stored as a .dll object), such that the other applications can include a graphical user interface (GUI), network and database technology (e.g., SQL®, Oracle®), and web-services. The process 100 can be installed as a rich client application (i.e., network access is not required), or as thin client such as within a browser (i.e., network access is required).

At stage 102, the genotype pattern information is received. The genotype patterns are based on the genotype for one or more (e.g., 2, 3, 4, etc) metabolic genes. The metabolic genes include, but are not limited to, fatty acid binding protein 2 (FABP2); peroxisome proliferator-activated receptor-gamma (PPARG); beta-2 adrenergic receptor (ADRB2); and beta-3 adrenergic receptor (ADRB3). An individual's genetic polymorphism pattern with respect to one or more of these genes reveals an individual's metabolic genotype. More preferably, an individual's metabolic genotype may be determined by identifying that individual's genetic polymorphism pattern with respect to one or more (i.e., 2, 3, 4, or 5) of the FABP2 (rs1799883), PPARG (rs1801282) locus, ADRB3 (rs4994) locus, ADRB2 (rs1042713) locus, and/or ADRB2 (rs1042714) locus.

The test facility 26 and the computers 28 can be configured to determine a genotype pattern for the user 22 based on a genetic sample. The correlations between the genetic polymorphism patterns and one or more of the metabolic genes are listed in table 1.

TABLE 1
Individual Composite Genotypes and Risk Patterns

| | | | | | | Composite |
|---|---|---|---|---|---|---|
| Genotype | WM Panel Genotypes AND Interpretations | | | | | Genotype |
| ID# | FABP2 | PPARG | ADRB3 | ADRB2-1 | ADRB2-2 | Pattern |
| 1 | 54Thr/*^{†} | 12Pro/Pro^{†} | 64Arg/*^{‡} | 16Gly/*^{‡} | 27Glu/* | Pattern # 5 |
| 2 | 54Thr/*^{†} | 12Pro/Pro^{†} | 64Arg/*^{‡} | 16Gly/*^{‡} | 27Gln/Gln | Pattern # 5 |
| 3 | 54Thr/*^{†} | 12Pro/Pro^{†} | 64Arg/*^{‡} | 16Arg/Arg | 27Glu/* | Pattern # 5 |
| 4 | 54Thr/*^{†} | 12Pro/Pro^{†} | 64Arg/*^{‡} | 16Arg/Arg | 27Gln/Gln | Pattern # 5 |
| 5 | 54Thr/*^{†} | 12Pro/Pro^{†} | 64Trp/Trp | 16Gly/*^{‡} | 27Glu/* | Pattern # 5 |
| 6 | 54Thr/*^{†} | 12Pro/Pro^{†} | 64Trp/Trp | 16Gly/*^{‡} | 27Gln/Gln | Pattern # 5 |
| 7 | 54Thr/*^{†} | 12Pro/Pro^{†} | 64Trp/Trp | 16Arg/Arg | 27Glu/* | Pattern # 2 |
| 8 | 54Thr/*^{†} | 12Pro/Pro^{†} | 64Trp/Trp | 16Arg/Arg | 27Gln/Gln | Pattern # 2 |
| 9 | 54Thr/*^{††} | 12Ala/*^{††} | 64Arg/*^{‡} | 16Gly/*^{‡} | 27Glu/*^{††} | Pattern # 6 |
| 10 | 54Thr/*^{††} | 12Ala/*^{††} | 64Arg/*^{‡} | 16Gly/*^{‡} | 27Gln/Gln | Pattern # 6 |
| 11 | 54Thr/*^{††} | 12Ala/*^{††} | 64Arg/*^{‡} | 16Arg/Arg | 27Glu/*^{††} | Pattern # 6 |
| 12 | 54Thr/*^{††} | 12Ala/*^{††} | 64Arg/*^{‡} | 16Arg/Arg | 27Gln/Gln | Pattern # 6 |
| 13 | 54Thr/*^{††} | 12Ala/*^{††} | 64Trp/Trp | 16Gly/*^{‡} | 27Glu/*^{††} | Pattern # 6 |
| 14 | 54Thr/*^{††} | 12Ala/*^{††} | 64Trp/Trp | 16Gly/*^{‡} | 27Gln/Gln | Pattern # 6 |
| 15 | 54Thr/*^{††} | 12Ala/*^{††} | 64Trp/Trp | 16Arg/Arg | 27Glu/* | Pattern # 3 |
| 16 | 54Thr/*^{††} | 12Ala/*^{††} | 64Trp/Trp | 16Arg/Arg | 27Gln/Gln | Pattern # 3 |
| 17 | 54Ala/Ala | 12Pro/Pro | 64Arg/*^{‡} | 16Gly/*^{‡} | 27Glu/*^{††} | Pattern # 6 |
| 18 | 54Ala/Ala | 12Pro/Pro | 64Arg/*^{‡} | 16Gly/*^{‡} | 27Gln/Gln | Pattern # 4 |
| 19 | 54Ala/Ala | 12Pro/Pro | 64Arg/*^{‡} | 16Arg/Arg | 27Glu/*^{††} | Pattern # 6 |
| 20 | 54Ala/Ala | 12Pro/Pro | 64Arg/*^{‡} | 16Arg/Arg | 27Gln/Gln | Pattern # 4 |
| 21 | 54Ala/Ala | 12Pro/Pro | 64Trp/Trp | 16Gly/*^{‡} | 27Glu/*^{††} | Pattern # 6 |
| 22 | 54Ala/Ala | 12Pro/Pro | 64Trp/Trp | 16Gly/*^{‡} | 27Gln/Gln | Pattern # 4 |
| 23 | 54Ala/Ala | 12Pro/Pro | 64Trp/Trp | 16Arg/Arg | 27Glu/*^{††} | Pattern # 3 |
| 24 | 54Ala/Ala | 12Pro/Pro | 64Trp/Trp | 16Arg/Arg | 27Gln/Gln | Pattern # 1 |
| 25 | 54Ala/Ala | 12Ala/*^{††} | 64Arg/*^{‡} | 16Gly/*^{‡} | 27Glu/*^{††} | Pattern # 6 |
| 26 | 54Ala/Ala | 12Ala/*^{††} | 64Arg/*^{‡} | 16Gly/*^{‡} | 27Gln/Gln | Pattern # 6 |
| 27 | 54Ala/Ala | 12Ala/*^{††} | 64Arg/*^{‡} | 16Arg/Arg | 27Glu/*^{††} | Pattern # 6 |
| 28 | 54Ala/Ala | 12Ala/*^{††} | 64Arg/*^{‡} | 16Arg/Arg | 27Gln/Gln | Pattern # 6 |
| 29 | 54Ala/Ala | 12Ala/*^{††} | 64Trp/Trp | 16Gly/*^{‡} | 27Glu/*^{††} | Pattern # 6 |
| 30 | 54Ala/Ala | 12Ala/*^{††} | 64Trp/Trp | 16Gly/*^{‡} | 27Gln/Gln | Pattern # 6 |
| 31 | 54Ala/Ala | 12Ala/*^{††} | 64Trp/Trp | 16Arg/Arg | 27Glu/*^{††} | Pattern # 3 |
| 32 | 54Ala/Ala | 12Ala/*^{††} | 64Trp/Trp | 16Arg/Arg | 27Gln/Gln | Pattern # 3 |

^{†}indicates PPARG AND FABP2 is a composite genotype which leads to a “Responsive to Fat Restriction” category for weight management goals
^{‡}indicates a genotype that leads to a “Less Responsive to Exercise” determination
^{††}indicates the composite PPARG, ADRB2, OR PPARG + FABP2 genotypes which will lead to a “Responsive to Carbohydrate Restriction” category for weight management goals

At stage 104, if the received genotype pattern information corresponds to pattern #1, then wellness information regarding a balanced diet 106 and moderate exercise 108 is provided. In general, individuals with a metabolic genotype that is responsive to a balanced diet or calorie restrictive diet 106 (i.e. a balance of fat and carbohydrate intake) show no consistent need for a low fat or low carbohydrate diet. In these individuals key biomarkers, such as body weight, body fat, and plasma lipid profile, respond well to a diet balanced in fat and carbohydrate. For individuals with this genetic pattern who are interested in losing weight, a balanced diet restricted in calories has been found to promote weight loss and a decrease in body fat.

A calorie restricted diet or balanced diet refers to a diet that is restricts total calories consumed to below an individual's weight maintenance level (WML), regardless of any preference for a macronutrient. A balanced diet or calorie restricted diet seeks to reduce the overall caloric intake of an individual by, for example, reducing the total caloric intake of an individual to below that individual's WML without a particular focus on restricting the calories consumed from any particular macronutrient. Thus, according to some embodiments, a balanced diet may be expressed as a percentage of an individual's WML. For example, a balanced diet is a diet that comprises a total caloric intake of between about 50% to about 100% WML. According to some embodiments, a balanced diet is a diet that comprises a total caloric intake of less than 100% (e.g., less than about 99%, 97%, 95%, 90%, 85%, 80%, 75%, 70%, 65%, 60%, 55%) of WML. Within this framework, a balanced diet achieves a healthy or desired balance of macronutrients in the diet and may be: low fat; low saturated fat; low carbohydrate; low fat and low carbohydrate; or low saturated fat and low carbohydrate. For example, a diet may be a low fat, calorie restricted diet (where low fat has the meaning as provided hereinabove). A diet may be a low carbohydrate, calorie restricted diet (where low carbohydrate has the meaning as provided hereinabove). A diet may be a balanced, calorie restricted diet (e.g., relative portions of macronutrients may vary where the total calories consumed is below the WML).

A typical balanced or calorie restricted diet provides 55% of calories from carbohydrates, 20% of calories from protein, and 25% of calories from fat. Exemplary third party diet plans based on a balanced diet include the Best Life Diet, a Mediterranean Diet, the Sonoma Diet, the Volumetrics Eating Plan (e.g., the Jenny Craig program), the Nutrisystem program, and a Weight Watchers Diet.

Typical moderate exercise information provided at stage 108 would include a routine comprising 2.5 hours (150 minutes) of moderate-intensity activity per week. Moderate-intensity activities are defined as approximately 3.0 to 5.9 METs, wherein an MET is equal to 1 calorie/kg body mass/hour. Examples of moderate-intensity include walking briskly, ballroom dancing, general gardening and water aerobics.

At stage 110, if the received genotype pattern information corresponds to pattern #2, then wellness information regarding a low-fat diet 112 and moderate exercise 108 is provided. In general, low-fat diet 112 refers to a diet that provides between about 10% to less than about 40% of total calories from fat. According to some embodiments, a low fat diet refers to a diet that provides no more than about 35 percent (e.g., no more than about 19%, 21%, 23%, 22%, 24%, 26%, 28%, 33%, etc) of total calories from fat. According to some embodiments, a low fat diet refers to a diet that provides no more than about 30 percent of total calories from fat. According to some embodiments, a low fat diet refers to a diet that provides no more than about 25 percent of total calories from fat. According to some embodiments, a low fat diet refers to a diet that provides no more than about 20 percent of total calories from fat. According to some embodiments, a low fat diet refers to a diet that provides no more than about 15 percent of total calories from fat. According to some embodiments, a low fat diet refers to a diet that provides no more than about 10 percent of total calories from fat.

According to some embodiments, a low fat diet refers to a diet that is between about 10 grams and about 60 grams of fat per day. According to some embodiments, a low fat diet refers to a diet that is less than about 50 grams (e.g., less than about 10, 25, 35, 45, etc) grams of fat per day. According to some embodiments, a low fat diet refers to a diet that is less than about 40 grams of fat per day. According to some embodiments, a low fat diet refers to a diet that is less than about 30 grams of fat per day. According to some embodiments, a low fat diet refers to a diet that is less than about 20 grams of fat per day.

Fats contain both saturated and unsaturated (monounsaturated and polyunsaturated) fatty acids. According to some embodiments, reducing saturated fat to less than 10 percent of calories is a diet low in saturated fat. According to some embodiments, reducing saturated fat to less than 15 percent of calories is a diet low in saturated fat. According to some embodiments, reducing saturated fat to less than 20 percent of calories is a diet low in saturated fat. Examples of low-fat diets include Life Choice Diet (Ornish Diet), Pritikin Diet, and many of the Heart Healthy diets.

At stage 114, if the received genotype pattern information corresponds to pattern #3, then wellness information regarding a low carbohydrate diet 116 and moderate exercise 108 is provided. Generally, a low carbohydrate (CHO) diet refers to a diet that provides between about 20% to less than about 50% of total calories from carbohydrates. According to some embodiments, a low carbohydrate (CHO) diet refers to a diet that provides no more than about 50 percent (e.g., no more than about 20%, 25%, 30%, 35%, 40%, 45%, etc) of total calories from carbohydrates. According to some embodiments, a low carbohydrate diet refers to a diet that provides no more than about 45 percent of total calories from carbohydrates. According to some embodiments, a low carbohydrate diet refers to a diet that provides no more than about 40 percent of total calories from carbohydrates. According to some embodiments, a low carbohydrate diet refers to a diet that provides no more than about 35 percent of total calories from carbohydrates.

According to some embodiments, a low carbohydrate diet refers to a diet that provides no more than about 30 percent of total calories from carbohydrates. According to some embodiments, a low carbohydrate diet refers to a diet that provides no more than about 25 percent of total calories from carbohydrates. According to some embodiments, a low carbohydrate diet refers to a diet that provides no more than about 20 percent of total calories from carbohydrates.

A low carbohydrate (CHO) diet can be a diet that restricts the amount of grams of carbohydrate in a diet such as a diet of from about 20 to about 250 grams of carbohydrates per day. According to some embodiments, a low carbohydrate diet comprises no more than about 220 (e.g., no more than about 40, 70, 90, 110, 130, 180, 210, etc) grams of carbohydrates per day. According to some embodiments, a low carbohydrate diet comprises no more than about 200 grams of carbohydrates per day. According to some embodiments, a low carbohydrate diet comprises no more than about 180 grams of carbohydrates per day. According to some embodiments, a low carbohydrate diet comprises no more than about 150 grams of carbohydrates per day. According to some embodiments, a low carbohydrate diet comprises no more than about 130 grams of carbohydrates per day. According to some embodiments, a low carbohydrate diet comprises no more than about 100 grams of carbohydrates per day. According to some embodiments, a low carbohydrate diet comprises no more than about 75 grams of carbohydrates per day. Examples of low carbohydrate diets include the Atkins Diet, Glycemic Impact Diet, South Beach Diet, Sugar Busters Diet, and the Zone Diet.

At stage 118, if the received genotype pattern information corresponds to pattern #4, then wellness information regarding a balanced diet 106 and vigorous exercise 120 is provided. In general, individuals with a metabolic genotype that is less responsive to exercise are less able to break down body fat for energy in response to exercise than those with the alternative genetic pattern. They tend to lose less weight and body fat than expected with moderate exercise. These individuals require more exercise to activate the breakdown of body fat for energy and weight loss. They must also maintain a consistent exercise program to keep the weight off. Typical vigorous exercise information provided at stage 120 would include a routine comprising greater than 13 METs per week of vigorous intensity activities. Vigorous intensity activities can be defined as 6 METs or greater, wherein an MET is equal to 1 calorie/kg body mass/hour. Examples of vigorous intensity include racewalking, jogging or running, hiking uphill (or with a heavy backpack), and swimming laps.

In general, both the moderate exercise information 108 and vigorous exercise information 120 suggest muscle strengthening activities that engage the major muscle groups should be included at least two days a week. These activities include weight training, push-ups, sit-ups, heavy gardening, or carrying heavy loads. The types and durations of both moderate 108 and vigorous 120 exercises can be adjusted based on individual results.

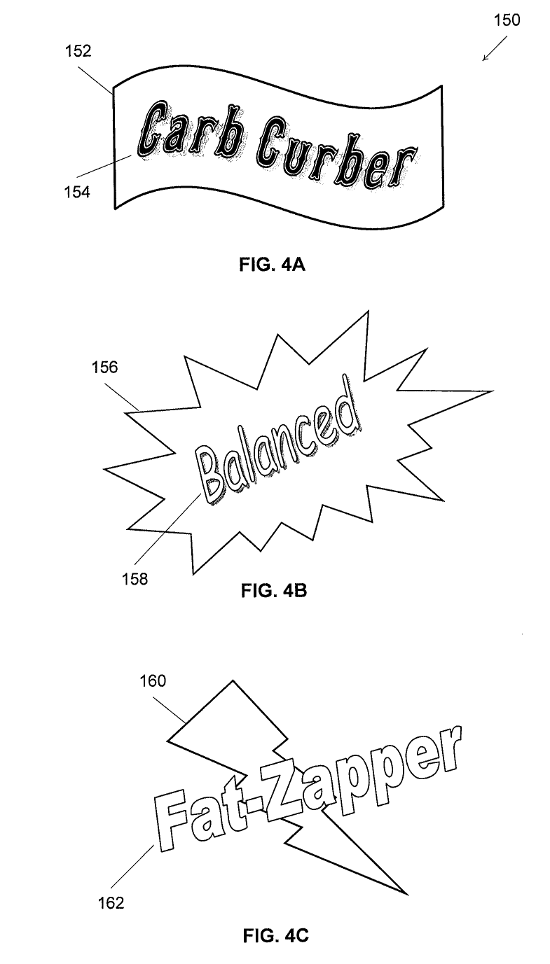

Referring to FIGS. 4A-C, with further reference to FIG. 3, an exemplary collection of logos 150 which correspond to at least one of the genotype patterns 12 is shown. The logos 150 are exemplary only and not limiting. The logos 150 may be altered, e.g. by having different designs including different text, names, fonts, shapes and colors.

In an embodiment, the genotype patterns 12 can be assigned at least one identifying logo 150. For example, the logos 150 can be used to help consumers identify nutritional products (e.g., food, vitamins, supplements) that are aligned genotype patterns (e.g., the balanced 106, low-fat 112, or low-carb 116 diets). Each of the six patterns can have a different logo. As an example, and not a limitation, the “pattern 3” 114 genotype can have a logo including a banner background 152 and tag line 154 of “Carb Curber.” Other genotype patterns can have different logos, including different colors, background shapes, taglines, or designs. In other examples, a logo for the “pattern 1” 104 genotype can include a burst shaped background 156 and a “Balanced” tagline 158. Similarly, as an example and not a limitation, a logo for the “pattern 2” genotype can have a lightning shaped background 160 and a “Fat-Zapper” 162 tagline. In an embodiment, a single logo can represent more than one genotype pattern (i.e., to indicate that a product is aligned to more than one genotype pattern). The objective of the logos is to provide a recognizable and familiar icon to assist consumers when purchasing wellness items such as food products and other dietary supplements.

In operation, in an embodiment, consumer food products and other dietary supplements can have an appropriate logo affixed, or otherwise printed, on them. For example, a prepared frozen meal consisting of a low-carbohydrate entree can include the “Carb Curber” logo on the packaging. In an embodiment, such as in a grocery store, a logo 150 can be part of a food display (e.g., shelf unit, produce bin, promotional display cart) to indicate that the nutritional value of the displayed foods comports with the requirements of the corresponding genetic pattern. In electronic embodiments, versions of the logos 150 can be icons that are displayed with appropriate foods, recipes, menu items, and shopping lists that are selected on a computer display (e.g., website, rich client application).

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. A method comprising transforming a dietary program of a person as a function of their genotype pattern.

2. A computerized method for providing wellness information to a user, comprising:
receiving a genetic sample;
determining a genotype pattern from the genetic sample;
generating, on at least one computer, a wellness report based on the genotype pattern; and
sending the wellness report to the user.

3. The computerized method of claim 2 wherein receiving the genetic sample includes receiving at least one consent form and a packet containing at least one genetic sample.

4. The computerized method of claim 3 wherein the packet and the consent form include matching bar-code information.

5. The computerized method of claim 2 wherein the genetic sample is a brush containing a tissue sample extracted from inside the user's cheek.

6. The computerized method of claim 2 wherein determining the genotype pattern comprises identifying the genotype with respect to at least one metabolic gene or gene variation.

7. The computerized method of claim 2 wherein determining the genotype pattern comprises identifying the genotype with respect to at least one of the following:
FABP2 (rs1799883);
PPARG (rs1801282);
ADRB3 (rs4994);
ADRB2 (rs1042713); or
ADRB2 (rs1042714).

8. The computerized method of claim 2 wherein the wellness report includes at least one logo representative of the genotype pattern.

9. The computerized method of claim 2 wherein the wellness report is provided to the user via a computer network.

10. An article of manufacture, comprising:
a nutritional product suitable to be consumed in a diet selected from the group consisting of a balanced diet, a low-fat diet, and a low-carb diet; and
a genotype pattern logo disposed on the nutritional product, wherein the genotype pattern logo represents a genotype that is used to predict a person's responsiveness to at least one of the diets.

11. The article of manufacture of claim 10 wherein the genotype pattern logo includes a background component and a tagline component.

12. The article of manufacture of claim 10 wherein the genotype pattern logo is associated with the person's genetic polymorphism pattern consisting of at least one metabolic gene or gene variant.

13. The article of manufacture of claim 10 wherein the genotype pattern logo is associated with the person's genetic polymorphism pattern with respect to one or more of the following:
FABP2 (rs1799883);
PPARG (rs1801282);
ADRB3 (rs4994);
ADRB2 (rs1042713); or
ADRB2 (rs1042714).

14. A method of providing genetic weight management information to a user, comprising:
obtaining a tissue sample from the user;
transforming the tissue sample into metabolic genotype pattern information; and
providing the genotype pattern information to the user.

15. The method of claim 14 further comprising generating a wellness report, wherein the wellness report includes diet and exercise recommendations.

16. The method of claim 14 wherein providing the genotype pattern information includes providing a genotype pattern logo.

17. The method of claim 14 wherein transforming the tissue sample includes identifying the user's genetic polymorphism pattern with respect to one or more of the group consisting of:
FABP2 (rs1799883);
PPARG (rs1801282);
ADRB3 (rs4994);
ADRB2 (rs1042713); and
ADRB2 (rs1042714).

18. The method of claim 14 further comprising providing the user a kit comprising: a consent form, a plurality of brushes, a drying stand, a packet, and a mailing envelope.

19. The method of claim 18 further comprising:
rubbing at least one brush against the inside of the user's cheek;
placing the brush in the drying stand for approximately 15 minutes to dry;
sealing the at least one dried brush into the packet; and
placing the packet into the mailing envelope.

20. The method of claim 18 wherein the consent form and the packet contain identical bar code information.

21. A computer network comprising:
a data storage device configured to store wellness information;
at least one display device configured to receive and display information from and to a user;
a processor programmed to:
receive a genotype pattern information from the user; and
provide wellness information as a function of the genotype pattern information to the user via the display.

22. The computer network of claim 21 wherein the wellness information includes recipes.

23. The computer network of claim 21 wherein the wellness information includes exercise recommendations.

24. The computer network of claim 21 wherein the wellness information includes recommendations for dietary supplements.
